(19) United States
(12) Patent Application Publication
(10) Pub. No.: US 2012/0272866 A1
(43) Pub. Date: Nov. 1, 2012

(54) Contact Lenses with Pores for High Oxygen Permeability and Manufacturing Method Thereof

(75) Inventors: Chung-Lin Wang, Taichung CIty (TW); Wei-Yuan Chen, Taichung City (TW)

(73) Assignee: LARGAN MEDICAL CO., LTD., Taichung City (TW)

(21) Appl. No.: 13/456,878

(22) Filed: Apr. 26, 2012

(30) Foreign Application Priority Data
Apr. 28, 2011 (TW) 100115284
Oct. 5, 2011 (TW) 100136180
Jan. 10, 2012 (TW) 101101009

(51) Int. Cl. A61L 31/14; B29D 11/00

(52) U.S. Cl. 106/122; 264/1.1

(57) ABSTRACT

Contact lenses with pores having high oxygen permeability and a manufacturing method thereof. The manufacturing method comprises the steps of uniformly mixing water-soluble salts and the formula of contact lenses having hydrophilic monomers to form a mixed formula; molding and forming the mixed formula to obtain contact lenses; dissolving and dialyzing the water-soluble salts by a water solution so as to form a plurality of pores with a predetermined diameter on a surface and in the interior of the contact lenses. The diameter of pore size has an allowable range between 0.03 μm and 0.05 μm such that the contact lenses with high oxygen permeability can be obtained.

### CROSS-REFERENCE TO RELATED APPLICATION

This application claims the benefit of Taiwan Patent Application Nos. 100115284, 100136180 and 101101009, respectively filed on Apr. 28, 2011, Oct. 5, 2011 and Jan. 10, 2012, in the Taiwan Intellectual Property Office, the disclosure of which is incorporated herein in its entirety by reference.

### FIELD OF THE INVENTION

The invention relates to contact lenses and a manufacturing method thereof, in particular to the contact lenses with pores for high oxygen permeability and the manufacturing method thereof.

### BACKGROUND OF THE INVENTION

Impaired vision and eye degeneration have troubled people for a long time, and especially after the revolution of industrial and the revolution of information, our society has transformed from an agricultural society into an industrial society. With the explosion of information, people nowadays have to spend more time and effort to access information. Under these conditions, the eyes play a more important role than ever, thus resulting in an increased rate of visual impairments due to the increased amount of workload, and the trend of visual degeneration also continues to rise. Whether it is vision correction surgery, regular eye care, or traditional glasses and contact lenses that are most commonly worn for the purpose of vision correction, there is an urgent demand for various technologies that contribute to vision improvement.

At present, it is very common practice for people to wear contact lenses to correct their vision since contact lenses do not have a heavy frame or thick lenses. The use of contact lenses will not create burdens to the users or affect the appearance of the users. In addition, for users that are in need of high powered glasses, when compared with wearing traditional frame glasses, contact lenses have lower impact on the proportional enlargement and reduction of the image, thereby providing the user with better vision.

However, surveys show that over 90% of contact lens users experience symptoms of corneal hypoxia. At the beginning, there are no significant symptoms for corneal hypoxia, and hence the health issues of the corneas are always easily ignored. The cornea provides approximately 75% of the eye's total refractive power. In order to maintain its transparency, corneal cells do not have oxygen conveyed by blood vessels, and thus it is necessary for the corneal cells to obtain oxygen directly from the air for metabolism. When a contact lens is worn on the cornea, contact lenses with low oxygen transmissibility will fail to provide sufficient oxygen to the cornea, blood vessels will be proliferated in order to supply oxygen to the oxygen-deficient cornea, causing more capillaries to be formed easily at the white of the eye.

In addition, when the cornea is oxygen deficient for long periods of time, blood vessels are proliferated on the cornea and serious problems such as damaged or deformed corneal endothelial cells and corneal edema may result. Corneal hypoxia also increases the number of bacteria attached onto the epithelial that causes corneal inflammation. If the contact lens user continues to ignore the problem of corneal hypoxia and delays in obtaining the required treatment, permanent damage to vision or even blindness may result.

To overcome the problem of contact lenses having low oxygen permeability, most inventions focus on changing the composition of the contact lenses in hopes of improving its oxygen permeability. For example, a flexible contact lens containing water was disclosed in U.S. Pat. No. 4,711,943, wherein the contact lens is made of a compound consisting of amides and siloxanes, but this compound reduces the wetness of the lens which causes the lens to be more easily contaminated by lipids. In addition, different manufacturing processes such as electrical discharge or laser irradiation may be used to increase the number of pores formed on the contact lenses in order to improve its oxygen permeability. However, these manufacturing processes have the drawback of reducing or losing the transparency of the resin, and thus imposing more limitations on the choice of materials used for making the contact lenses.

### SUMMARY OF THE INVENTION

In view of the foregoing problem of prior art, the purposes of this invention are providing a contact lens with pores having high oxygen permeability, and a manufacturing method in order to overcome the problem of corneal hypoxia caused by wearing the contact lenses for long periods of time.

For the purposes, the present invention provides a manufacturing method of contact lenses with pores having high oxygen permeability, comprising the steps of: uniformly mixing a water-soluble salt with a formula of the contact lenses having hydrophilic monomers to form a mixed formula; molding the mixed formula to form the contact lenses; and dissolving and dialyzing the water-soluble salt by a water solution, so as to form plurality of pores on the surface and in the interior of the contact lenses.

Preferably, the diameter of pore size has an allowable range between 0.01 μm and 50 μm.

Preferably, the water-soluble salt is a kind of sodium salt, potassium salt, calcium salt, magnesium salt, iron salt, zinc salt, copper salt or manganese salt.

Preferably, the concentration of the water-soluble salt falls within a range from 0.01 wt % to 30 wt % (percentage by weight) of the mixed formula.

Preferably, the concentration of the water-soluble salt falls within a range from 0.1 wt % to 10 wt % (percentage by weight) of the mixed formula.

Preferably, the hydrophilic monomers include a benzophenone compound.

Preferably, the formula of the contact lenses contains 2-hydroxyethyl methacrylate, glycerol monomethacrylate, propan-1,2,3-triol, ethylene glycol dimethacrylate, 2-hydroxy-2-methyl-phenyl-propane-1-one, phenylbis (2,4,6-trimethylbenzoyl)phosphine oxide, and 2-propenoic acid, or 2-methyl-1,1′-[(9,10-dihydro-9,10-dioxo-1,4-anthracenediyl)bis(imino-4,1-phenylene-2,1ethanediyl)]ester.

In addition, the present invention further provides a contact lens with pores having high oxygen permeability which is manufactured by the aforementioned manufacturing method.

In summation, the contact lenses with pores having high oxygen permeability and the manufacturing method in accordance with the present invention have one or more of the following advantages.

(1) In the manufacturing method of the contact lenses with pores having high oxygen permeability in accordance with the present invention, the pores of the contact lenses are formed by mixing a normally and physiologically metabolizable water-soluble salt with a formula of contact lenses with hydrophilic monomers, and then dissolving and dialyzing the water-soluble salts by a water solution so as to form a plurality of pores on the surface and in the interior of the contact lenses. Compared with the traditional methods, the manufacturing method of the present invention is simple and easy, and thus requiring no complicated laser manufacturing process to achieve the pore formation with the same effect. The present invention can achieve the effect of high oxygen permeability in a simple and easy manner.

(2) The manufacturing method of the contact lenses with pores having high oxygen permeability in accordance with the present invention adopts low-cost water-soluble salts and hydration of hydrophilic monomers to produce the pores, so that the manufacturing cost is lower than that of the laser irradiation process.

(3) In the manufacturing method of the contact lenses with pores having high oxygen permeability in accordance with the present invention, the increased oxygen permeability of the contact lenses is not caused by changing the composition of the contact lenses, so that the wetness of the contact lenses can be maintained, and contaminations caused by contaminants such as lipids can be avoided.

### BRIEF DESCRIPTION OF THE DRAWINGS

FIG. 1 shows a flow chart of a manufacturing method of contact lenses with high oxygen permeability in accordance with a preferred embodiment of the present invention;

FIG. 2 shows a microscopic view of contact lenses of the present invention before a hydration takes place;

FIG. 3 shows a microscopic view of contact lenses of the present invention after a hydration takes place;

FIG. 4 shows a histogram comparing the oxygen permeability of contact lenses manufactured with adding sodium chloride and the oxygen permeability of contact lenses manufactured without adding sodium chloride.

### DESCRIPTION OF THE PREFERRED EMBODIMENTS

### Embodiment 1

The invention illustrates a manufacturing method of contact lenses with high oxygen permeability.

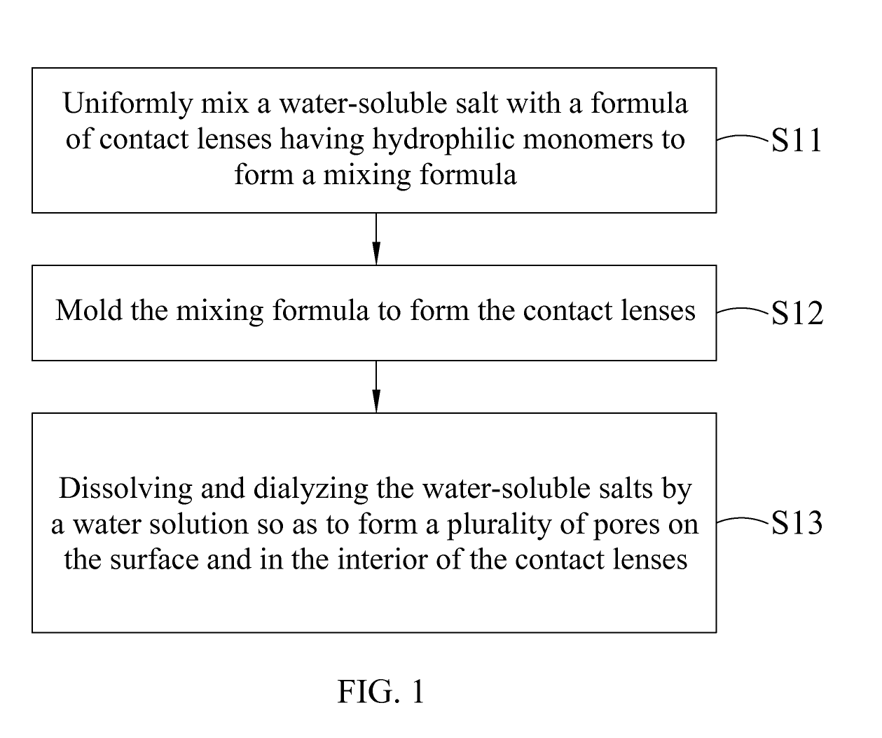

With reference to FIG. 1 for a flow chart of a manufacturing method of contact lenses with pores for high oxygen permeability in accordance with a preferred embodiment of the present invention, the manufacturing method comprises the following steps:

S11: Uniformly mixing a water-soluble salt with a formula of contact lenses having hydrophilic monomers to form a mixed formula.

S12: Molding the mixed formula to form the contact lenses.

S13: Dissolving and dialyzing the water-soluble salts by a water solution so as to form a plurality of pores on the surface and in the interior of the contact lenses.

Wherein, the diameter of pores size of each of the pores has an allowable range between 0.01 μm and 50 μm, and the water-soluble salt has a concentration falling within a range from 0.01 wt % to 30 wt % (percentage by weight of the mixed formula), and the concentration preferably falls within an allowable range between 0.1 wt % and 10 wt %.

Preferably, the water-soluble salt is a kind of sodium salt, potassium salt, calcium salt, magnesium salt, iron salt, zinc salt, copper salt or manganese salt. The salt can be a physiological metabolizable salt, so that the salt is not poisonous nor become a burden to the user's body. The formula of contact lenses includes hydrophilic monomers, 2-hydroxyethyl methacrylate, glycerol monomethacrylate, propan-1,2,3-triol, ethylene, glycol dimethacrylate, 2-hydroxy-2-methyl-phenyl-propane-1-one, phenylbis(2,4,6-trimethylbenzoyl) phosphine oxide, and 2-propenoic acid, or 2-methyl-1,1′-[(9,10-dihydro-9,10-dioxo-1,4-anthracenediyl)bis(imino-4,1-phenylene-2,1ethanediyl)]ester.

The hydrophilic monomers include a benzophenone compound with different functional groups and having the following chemical structure:

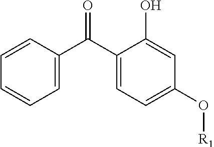

R₁ can be ethylacrylate (—C—C—COO—C═C) or methacrylate (—O—CO—CC═C). If R₁ is ethylacrylate, the hydrophilic monomer is 2-(4-benzoyl-3-hydroxyphenoxy)ethyl acrylate. If R₁ is methacrylate, the hydrophilic monomer is 4-methacryloxy-2-hydroxybenzophenone. The chemical structures of 2-(4-benzoyl-3-hydroxyphenoxy)ethyl acrylate and 4-methacryloxy-2-hydroxybenzophenone are shown by the following Compound 1 and Compound 2 respectively.

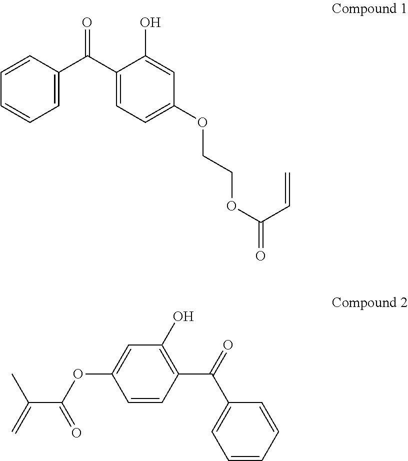

In the formula of the contact lenses, the Compound 1 has a percentage by weight falling within a range from 0.001 wt % to 10 wt % and Compound 2 has a percentage by weight falling within a range from 0.001 wt % to 10 wt %. The 2-hydroxyethyl methacrylate has a percentage by weight falling within a range from 0.01 wt % to 99 wt %, the glycerol monomethacrylate has a percentage by weight falling within a range from 0.01 wt % to 99 wt %, the propan-1,2,3-triol has a percentage by weight falling within a range from 0.01 wt % to 50 wt %, the ethylene glycol dimethacrylate has a percentage by weight falling within a range from 0.01 wt % to 30 wt %, the 2-hydroxy-2-methyl-phenyl-propane-1-one has a percentage by weight falling within a range from 0.01 wt % to 10 wt %, the phenylbis (2,4,6-trimethylbenzoyl)phosphine oxide has a percentage by weight falling within a range from 0.01 wt % to 10 wt %, and the 2-propenoic acid, 2-methyl-1,1′-[(9,10-dihydro-9,10-dioxo-1,4-anthracenediyl)bis(imino-4,1-phenylene-2,1-ethanediyl)]ester has a percentage by weight falling within a range from 0.0001 wt % to 1 wt %.

Preferably, the Compound 1 has a percentage by weight falling within a range from 0.01 wt % to 2 wt %, and the Compound 2 has a percentage by weight falling within a range from 0.01 wt % to 2 wt %. The 2-hydroxyethyl methacrylate has a percentage by weight falling within a range from 20 wt %-55 wt %, the glycerol monomethacrylate has a percentage by weight falling within a range from 37 wt % to 48 wt %, the propan-1,2,3-triol has a percentage by weight falling within a range from 1 wt % to 35 wt %, the ethylene glycol dimethacrylate has a percentage by weight falling within a range from 0.01 wt % to 10 wt %, the 2-hydroxy-2-methyl-phenyl-propane-1-one has a percentage by weight falling within a range from 0.1 wt % to 1.5 wt %, the phenylbis (2,4,6-trimethylbenzoyl)phosphine oxide has a percentage by weight falling within a range from 0.01 wt % to 1 wt %, and the 2-propenoic acid, 2-methyl-1,1′-[(9,10-dihydro-9,10-dioxo-1,4-anthracenediyl)bis(imino-4,1-phenylene-2,1-ethanediyl)]ester has a percentage by weight falling within a range from 0.001 wt % to 1 wt %.

Preferably, the Compound 1 has a percentage by weight falling within a range from 0.262 wt % to 0.69 wt %, and the Compound 2 has a percentage by weight falling within a range from 0.587 wt % to 0.972 wt %. The 2-hydroxyethyl methacrylate has a percentage by weight falling within a range from 29.1 wt % to 46.35 wt %; the glycerol monomethacrylate has a percentage by weight falling within a range from 40.74 wt % to 44.29 wt %; the propan-1,2,3-triol has a percentage by weight falling within a range from 8.73 wt % to 26.265 wt %; the ethylene glycol dimethacrylate has a percentage by weight falling within a range from 0.485 wt % to 1.03 wt %; the 2-hydroxy-2-methyl-phenyl-propane-1-one has a percentage by weight falling within a range from 0.388 wt % to 0.773 wt %; the phenylbis (2,4,6-trimethylbenzoyl)phosphine oxide has a percentage by weight falling within a range from 0.039 wt % to 0.155 wt %; and the 2-propenoic acid, 2-methyl-1,1′-[(9,10-dihydro-9,10-dioxo-1,4-anthracenediyl)bis(imino-4,1-phenylene-2,1-ethanediyl)]ester has a percentage by weight falling within a range from 0.005 wt % to 0.02 wt %.

### Embodiment 2

The invention is further provided for illustrating as follows:

To enable persons ordinarily skilled in the art to achieve the invention according to the preferred embodiments, the following preferred embodiments of the invention are disclosed for achieving the manufacturing method of the contact lenses with pores having high oxygen permeability. However, it is noteworthy to notice that the data for each composition of the present invention are provided for illustrating the technical characteristics of the invention only, but not intended for limiting the scope of the present invention.

In a preferred embodiment, small-diameter water-soluble salts (such as sodium chloride with a diameter of approximately 0.03-0.05 μm is mixed with a formula of contact lenses in the ratio of 49:1 (not limited to this ratio only), and each composition used in the formula of contact lenses is listed in Table 1.

TABLE 1

| Composition | wt % |
|---|---|
| Compound 1 | 0.45 |
| Compound 2 | 0.605 |
| 2-hydroxyethyl methacrylate | 45 |
| Glycerol monomethacrylate | 43 |
| Propan-1,2,3-triol | 10 |
| Ethylene glycol dimethacrylate | 0.5 |
| 2-hydroxy-2-methyl-phenyl-propane-1-one | 0.4 |
| Phenylbis (2,4,6-trimethylbenzoyl) phosphine oxide | 0.04 |
| 2-propenoic acid,2-methyl-1,1′-[(9,10-dihydro-9,10- | 0.005 |
| dioxo1,4-anthracenediyl)bis(imino-4,1-phenylene-2,1- | |
| ethanediyl)] ester | |

In addition to the aforementioned compositions, the formula of contact lenses of the present invention can further comprise the steps of mixing the contents as listed in Example 1 or Example 2 in Table 2 with the water-soluble salt, and the resulting mixed formula is used for the molding and forming a contact lens.

TABLE 2

| | Example 1 | Example 2 |
|---|---|---|
| Composition | (wt %) | (wt %) |
| Compound 1 | 0.27 | 0.67 |
| Compound 2 | 0.81 | 0.90 |
| 2-hydroxyethyl methacrylate | 45 | 30 |
| Glycerol monomethacrylate | 43 | 42 |
| Propan-1,2,3-triol | 9 | 25.5 |
| Ethylene glycol dimethacrylate | 1 | 0.5 |
| 2-hydroxy-2-methyl-phenyl-propane-1-one | 0.75 | 0.6 |
| Phenylbis(2,4,6-trimethylbenzoyl)phosphine | 0.15 | 0.1 |
| oxide | | |
| 2-propenoic acid,2-methyl-1,1′-[(9,10-dihydro- | 0.02 | 0.005 |
| 9,10-dioxo-1,4-anthracenediyl)bis(imino-4,1- | | |
| phenylene-2,1-ethanediyl)] ester | | |

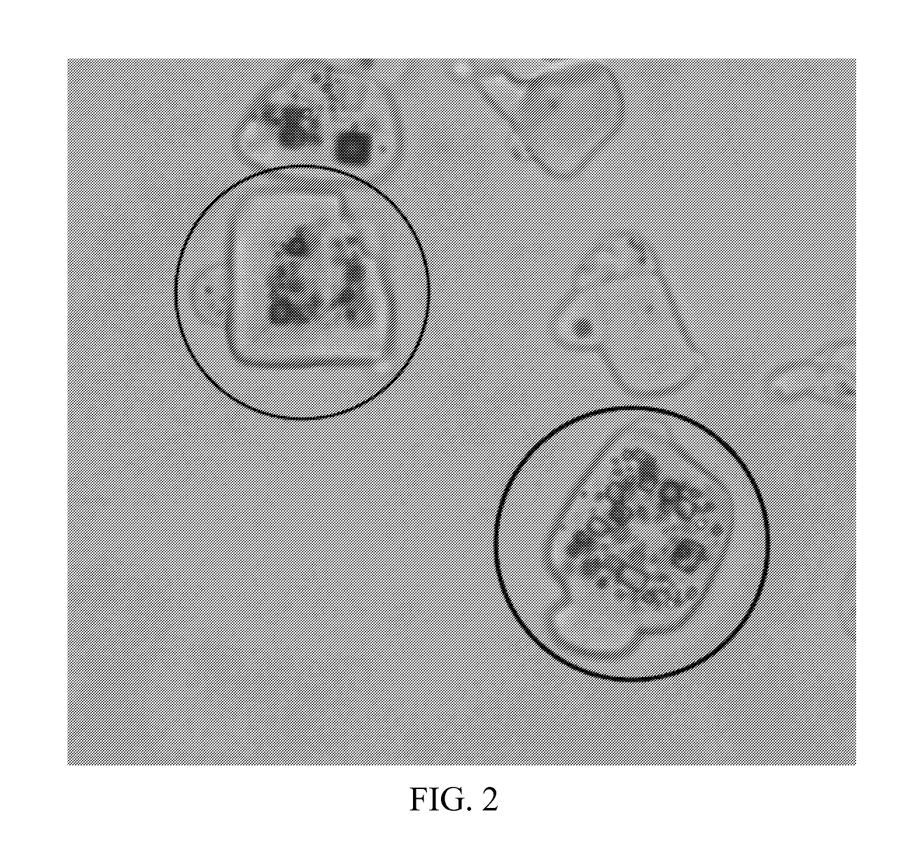

After the water-soluble salt is mixed with the formula of contact lenses as shown in Table 1 to obtain a mixed formula, the mixed formula is applied into a mold in the shape of the contact lenses, and a general manufacturing process of the contact lenses is used to form the contact lenses as shown in FIG. 2. In FIG. 2, the contact lenses contain salt particles (as indicated by the circles in FIG. 2).

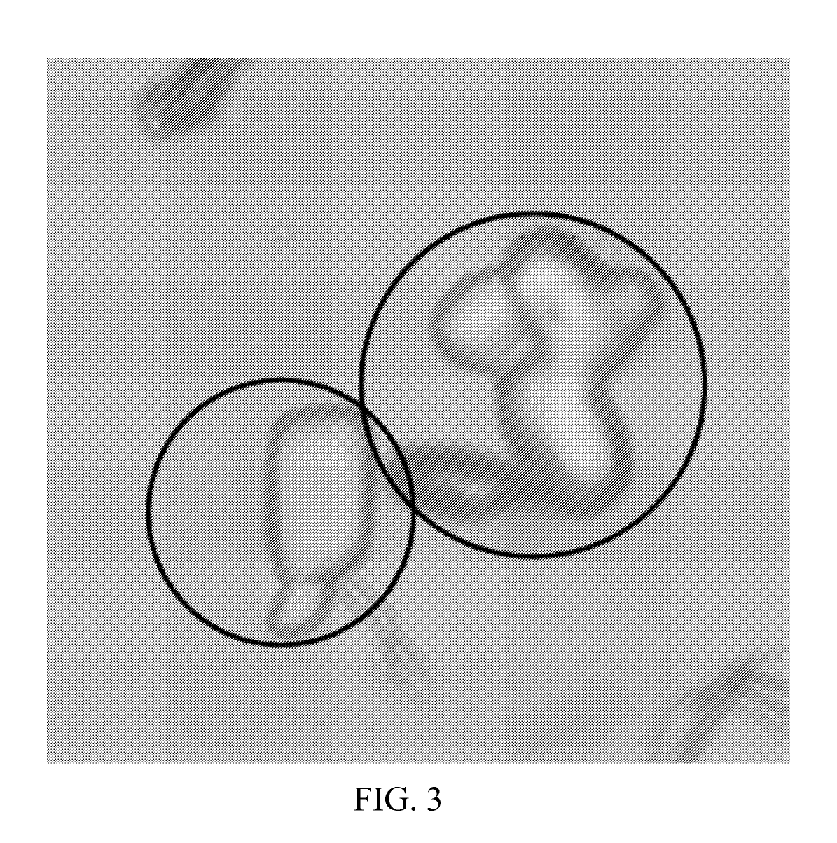

Further, sufficient water solution (such as deionized water) is used to dissolve and dialyze the water-soluble salts, the contact lenses will have a plurality of pores remained thereon (as indicated by the circles in FIG. 3), and the size of the pores can be adjusted by grinding and sieving the water-soluble salt. More specifically, the water-soluble salt can be processed by processes such as concentrating, filtering, crushing, grading, drying or spray drying to control the particle diameter of the water-soluble salt between 0.01 μm and 5 μm. Preferably, the diameter of pore size has an allowable range between 0.03 μm and 0.05 μm which is close to the size of the particle diameter of the originally used salt, and the pores formed on the surface and in the interior improve the possibility of exchanging oxygen, so as to improve the oxygen permeability of the contact lenses.

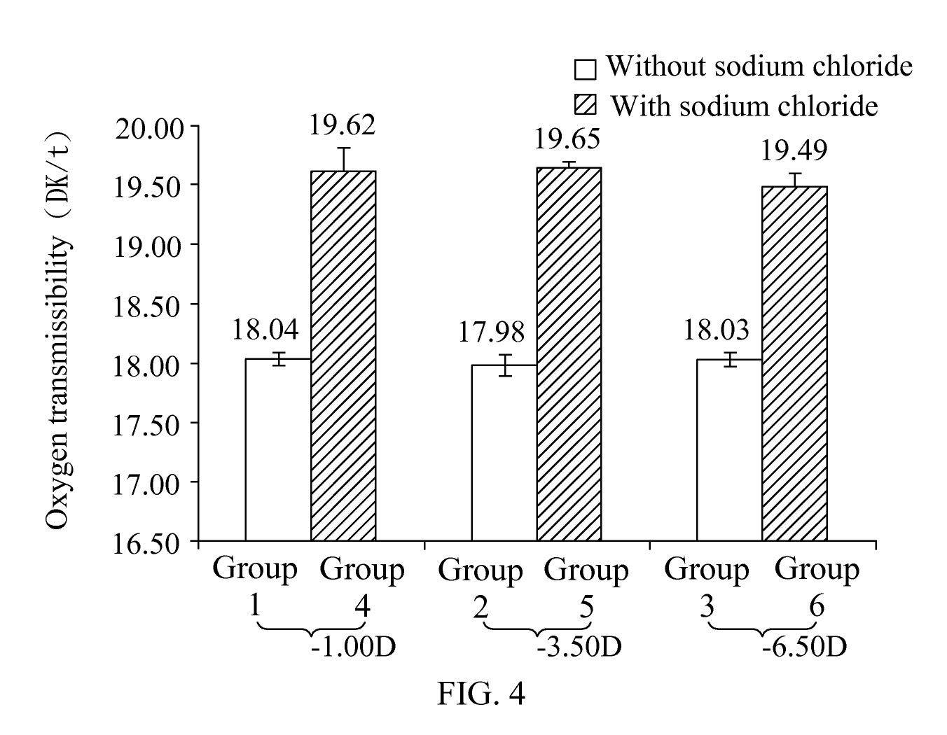

These results and data of the oxygen permeability of the contact lenses are listed below. Wherein, this preferred embodiment adopts contact lenses having a refractive power (in a unit of diopter) of −1.00 D, −3.50 D and −6.50 D and without adding any sodium chloride for the test, and the testing groups are divided into six groups, wherein Groups 1 to 3 do not contain any added sodium chloride during the manufacturing process, and Groups 4 to 6 contain added sodium chloride (in a ratio of 99:1) as shown in Table 3. The measured oxygen permeability of each group of contact lenses is listed in Table 4, Table 5 and FIG. 4.

TABLE 3

| | | Whether or not sodium |
|---|---|---|
| | Diopter | chloride is added? |
| Group 1 (n = 4) | −1.00D | No |
| Group 2 (n = 4) | −3.50D | No |
| Group 3 (n = 4) | −6.50D | No |
| Group 4 (n = 4) | −1.00D | Yes |
| Group 5 (n = 4) | −3.50D | Yes |
| Group 6 (n = 4) | −6.50D | Yes |

TABLE 4

| | | |
|---|---|---|
| Oxygen transmissibility | Center thickness (t) | Oxygen |
| (DK) | (cm) | permeability (DK/t) |

Group 1

| | | |
|---|---|---|
| 25.53 | 0.0141 | 18.11 |
| 24.99 | 0.0139 | 17.98 |
| 26.16 | 0.0145 | 18.04 |
| 25.92 | 0.0144 | 18.02 |

| | |
|---|---|
| Mean | 18.04 |
| Standard deviation | 0.05 |

Group 2

| | | |
|---|---|---|
| 17.16 | 0.0095 | 18.06 |
| 16.83 | 0.0094 | 17.90 |
| 16.47 | 0.0092 | 17.90 |
| 17.52 | 0.0097 | 18.06 |

| | |
|---|---|
| mean | 17.98 |
| standard deviation | 0.09 |

Group 3

| | | |
|---|---|---|
| 11.91 | 0.0066 | 18.05 |
| 11.76 | 0.0065 | 18.09 |
| 11.31 | 0.0062 | 17.95 |
| 12.27 | 0.0068 | 18.04 |

| | |
|---|---|
| Mean | 18.03 |
| Standard deviation | 0.06 |

TABLE 5

| | | |
|---|---|---|
| Oxygen transmissibility | Center thickness (t) | Oxygen |
| (DK) | (cm) | permeability (DK/t) |

Group 4

| | | |
|---|---|---|
| 26.73 | 0.0136 | 19.65 |
| 26.88 | 0.0138 | 19.48 |
| 26.64 | 0.0134 | 19.88 |
| 26.85 | 0.0138 | 19.46 |

| | |
|---|---|
| Mean | 19.62 |
| Standard deviation | 0.1 |

Group 5

| | | |
|---|---|---|
| 17.94 | 0.0091 | 19.71 |
| 18.03 | 0.0092 | 19.6 |
| 17.88 | 0.0091 | 19.65 |
| 17.85 | 0.0091 | 19.62 |

| | |
|---|---|
| Mean | 19.65 |
| Standard deviation | 0.05 |

Group 6

| | | |
|---|---|---|
| 12.06 | 0.0062 | 19.45 |
| 12.27 | 0.0063 | 19.48 |
| 12.21 | 0.0063 | 19.38 |
| 12.57 | 0.0064 | 19.64 |

| | |
|---|---|
| Mean | 19.49 |
| Standard deviation | 0.11 |

In Tables 4 and 5, the oxygen transmissibility (O₂ Transmissibility) is represented by a DK value (in a unit of [10⁻¹¹ (cm³O₂*cm²/sec*cm³*mmHg)], wherein, D is a diffusion coefficient indicating the diffusability of gas that passes through a material), which is also the moving speed of gas molecules in a substance; and K is a solubility coefficient indicating the level of oxygen solubility of the material, which is also the volume of gas soluble in a unit volume of material at a specific pressure. The oxygen permeability (O₂ Permeability) is equal to oxygen transmissibility/thickness and in the unit of [10⁻¹¹ (cm³O₂*cm/sec*cm³*mmHg)].

In Tables 4 and 5 and FIG. 4, unlike Groups 1, 2 and 3, the Groups 4, 5 and 6 have an increased oxygen permeability of 8.76%, 9.26% and 8.07% respectively. In other words, the sodium chloride and the formula of contact lenses having the hydrophilic monomers are mixed in the manufacturing process of the present invention to mold and form contact lenses, dissolving and dialyzing the water-soluble salts by a water solution, so as to produce the contact lenses with a plurality of pores formed on a surface and in the interior of the contact lenses, and the contact lenses with a higher oxygen permeability can be produced.

In summation of the description above, the manufacturing method of contact lenses with pores for high oxygen permeability in accordance with the present invention, the pores are formed on the contact lenses by mixing a human catabolized water-soluble salt with the formula of contact lenses having hydrophilic monomers, and then dissolving and dialyzing the water-soluble salts by a water solution. Compared with the traditional methods, the contact lenses of the present invention can have more pores which are also larger in diameter, if necessary, by adjusting the composition and the particle diameter of the water-soluble salt, the effects of improving oxygen permeability and avoiding the problem of corneal hypoxia can be achieved.

While the invention has been described by means of specific embodiments, numerous modifications and variations could be made thereto by those skilled in the art without departing from the scope and spirit of the invention set forth in the claims.

What is claimed is:

1. A manufacturing method of contact lenses with pores for high oxygen permeability, comprising the steps of:
uniformly mixing a water-soluble salt with a formula of the contact lenses having hydrophilic monomers to form a mixed formula;
molding the mixed formula to form the contact lenses; and
dissolving and dialyzing the water-soluble salt by a water solution, so as to form a plurality of pores on a surface and an interior of the contact lenses after the hydration takes place.

2. The manufacturing method of contact lenses with pores having high oxygen permeability as recited in claim 1, wherein the diameter of pores size of each of the pores has an allowable range between 0.01 μm and 50 μm.

3. The manufacturing method of contact lenses with pores having high oxygen permeability as recited in claim 1, wherein the water-soluble salt is one selected from a collection of a sodium salt, a potassium salt, a calcium salt, a magnesium salt, an iron salt, a zinc salt, a copper salt and a manganese salt.

4. The manufacturing method of contact lenses with pores having high oxygen permeability as recited in claim 3, wherein the water-soluble salt has a concentration expressed in term of percentage by weight of the mixed formula and ranging from 0.01 wt % to 30 wt %.

5. The manufacturing method of contact lenses with pores having high oxygen permeability as recited in claim 4, wherein the water-soluble salt has a concentration expressed in term of percentage by weight of the mixed formula and ranging from 0.1 wt % to 10 wt %.

6. The manufacturing method of contact lenses with pores having high oxygen permeability as recited in claim 1, wherein the hydrophilic monomers comprise a benzophenone compound.

7. The manufacturing method of contact lenses with pores having high oxygen permeability as recited in claim 6, wherein the formula of contact lenses comprises: 2-hydroxyethyl methacrylate; glycerol monomethacrylate; propan-1,2,3-triol; ethylene glycol dimethacrylate; 2-hydroxy-2-methyl-phenyl-propane-1-one; phenylbis (2,4,6-trimethylbenzoyl)phosphine oxide; and 2-propenoic acid, 2-methyl-1,1′-[(9,10-dihydro-9,10-dioxo-1,4-anthracenediyl)bis(imino-4,1-phenylene-2,1-ethanediyl)]ester.

8. A contact lens with pores having high oxygen permeability, manufactured by the manufacturing method as recited in claim 1.

9. A contact lens with pores having high oxygen permeability, manufactured by the manufacturing method as recited in claim 2.

10. A contact lens with pores having high oxygen permeability, manufactured by the manufacturing method as recited in claim 3.

11. A contact lens with pores having high oxygen permeability, manufactured by the manufacturing method as recited in claim 4.

12. A contact lens with pores having high oxygen permeability, manufactured by the manufacturing method as recited in claim 5.

13. A contact lens with pores having high oxygen permeability, manufactured by the manufacturing method as recited in claim 6.

14. A contact lens with pores having high oxygen permeability, manufactured by the manufacturing method as recited in claim 7.
